(19) United States
(12) Patent Application Publication
(10) Pub. No.: US 2005/0271739 A1
(43) Pub. Date: Dec. 8, 2005

(54) Methods and compositions for accelerating alcohol metabolism

(76) Inventor: Xiang H. Wang, Alpharetta, GA (US)

Correspondence Address: Zhaoyang Li;Squire, Sanders & Dempsey L.L.P., Suite 300, 1 Maritime Plaza, San Francisco, CA, 94111, US

(21) Appl. No.: 10/864,744

(22) Filed: Jun. 8, 2004

(51) Int. Cl. A61K 35/55; A61K 35/72

(52) U.S. Cl. 424/562; 424/195.16

(57) ABSTRACT

A composition and method of using thereof for accelerating alcohol metabolism are provided.

### BACKGROUND OF THE INVENTION

1. Field of the Invention

This invention generally relates to a composition for accelerating alcohol metabolism.

2. Description of the Background

Alcohol use is widespread throughout the world and has been throughout history. Consumption of alcoholic beverages in moderate amounts is an accepted societal practice. It is considered by many people to enhance the flavor and enjoy of food. Additionally, consumption of alcoholic beverages in moderate amounts is considered to provide some health benefits in terms of reduced stress and incidence of heart attack. It is also reported that in addition to having fewer heart attacks and strokes, moderate consumers of alcoholic beverages (beer, wine or distilled spirits or liquor) are generally less likely to suffer hypertension or high blood pressure, peripheral artery disease, Alzheimer's disease and the common cold.

However, drinking large amounts of alcohol can have very serious consequences. When alcohol beverages are consumed, the alcohol enters the stomach and is soon transported to the small intestine. From here the alcohol enters the blood stream via the portal vein and goes to the liver and every part of the body through the general circulation. The alcohol has free access to every cell in the body and exerts an influence on the central nerves system and brain. Alcohol increases dopamine activity, feeling pleasure and well being by slackening the brains controlling functions. The intensity of the effect of alcohol is directly related to the concentration of alcohol in the blood, also called “blood alcohol reading (BAR)”. Drinking a high volume of alcohol decreases one's sense of distinction, memory, concentration and reasoning. Table 1 lists the behavioral effects of blood alcohol reading (BAR).

TABLE 1
Behavioral Effects of Alcohol

| Lower BARs | Middle BARs | High BARs |
|---|---|---|
| (0.10-0.20%) | (0.03-0.08%) | (>0.20%) |
| Increases reaction times | Perceptual effects | Pensorimotor effects |
| Impairs gross motor | Decreases psychomotor | Anesthetic effects |
| function | performance | (0.30-0.40%) |
| Extreme sedation | Slower speech | Death; LD50 |
| | | (0.45-0.50%) |
| | Loss of consciousness | |

Excessive drinking causes serious health problems. The pathogenic effects of alcohol are very complex and have been attributed to the both alcohol and its metabolite intermediates: acetaldehyde and oxidative radicals, as shown in FIG. 1. Acetaldehyde is a reactive compound and can interact with thiol and amino groups of amino acids in proteins. Formation of acetaldehyde adducts with proteins may cause inhibition of that protein's function and/or cause an immune response. There is evidence that reactive aldehydic products resulting from ethanol metabolism and ethanol-induced oxidative stress play a pivotal role in the pathogenesis of alcoholic liver injury. In addition, reactive aldehydes and hydroxyl radicals are known for their ability to attack amino acid residues of proteins thereby forming both stable and unstable adducts with proteins and cellular constituents.

A number of compositions have been developed for reducing health damages caused by drinking with limited success. For example, U.S. Pat. No. 4,450,153 to Hopkins proposes a process and compositions suitable for the reduction of alcohol in the human blood supply to reduce the effect of alcohol consumption. Hopkins proposes to reduce the alcohol content in human blood by the administration of alcohol oxidase. U.S. Pat. No. 5,759,539 to Whitmire describes a method and formulations that accelerate ethanol elimination from the body. The formulations combine enzymes that oxidize alcohol to acetate, enzymes which regenerate NADH (nicotinamide adenine dinucleotide in its reduced form) to NAD (nicotinamide adenine dinucleotide), substrates which are rate limiting for the requisite enzymes, buffering agents which protect the enzymes against pH variations (e.g. low gastric pH), gastric acid sequestrants which block synthesis of gastric acid, and protease inhibiting agents and other agents which protect the active enzymes against proteolysis, carbohydrates which protect labile enzymes against bile salt inactivation.

U.S. Pat. No. 6,284,244 to Owades proposes a method for lowering the blood alcohol level by oral administration of an active dry yeast containing the enzyme alcohol dehydrogenase to a person before or concomitantly with the drinking of the alcoholic beverage to oxidize a portion of the alcohol while it is still in the stomach of the person. The alcohol dehydrogenase may be consumed as the purified enzyme, or more conveniently, by the ingestion of a natural source of the enzyme, such as active dry bakers, brewers, vintners and distillers yeast. According to Owades, ingesting active dry bakers yeast (the yeast most readily available commercially) or brewers, vintners or distillers yeast, just before, or during the drinking of an alcohol beverage, oxidizes a portion of the alcohol while still in the stomach, which results in a lower peak blood alcohol level, and also a lesser area under the curve of a plot of blood alcohol level vs. time. However, the action of the alcohol dehydrogenase on the alcohol is only in the stomach, so the alcohol dehydrogenase source must be ingested while the alcoholic beverage is still in the stomach. It will have no effect once the alcohol has left the stomach and entered the bloodstream, because the enzyme is destroyed by the acidity and proteolytic action in the stomach.

U.S. Pat. No. 4,877,601 to Wren provides a composition that contains a physiologically inert hydrophobic molecular sieve material, particularly a crystalline zeolite and a method to produce it in an edible form. The hydrophobic molecular sieve material has a pore size such as to permit the absorption of ethanol but the exclusion of other organic materials present in the blood or intestines. According to Wren, the administration of such molecular sieves, particularly hydrophobic zeolites, to human beings can be used for the treatment of the human body to lower the content of alcohol in the body. Such zeolites are prepared in a form suitable for administration by dispersion in an edible or physiologically acceptable base and particularly in dosage unit form having regard to the amount of alcohol to be absorbed.

U.S. Patent Application 20020006910 by Miamikov and Kashlinsky describes the use of compositions comprised of a sugar, L-glutamic acid, succinic acid, fumaric acid, ascorbic acid and aspartic acid to reduce drunkenness, remove alcohol intoxication and prevent hangover. Other methods and compositions drawn to reduce side effects of drinking include U.S. Pat. No. 4,857,523 to Lotsof drawn to oral administration of ibogaine and its salt for reducing alcohol dependency, U.S. Pat. No. 5,324,516 to Pek et al. drawn to a composition of fructose and an aqueous extract of pueraria flower, phaseoli radiati semen, and pinellia tubes for reducing blood alcohol concentration, and U.S. Pat. No. 6,485,758 to Mirza et al. drawn to using ephedrine (in a powdered form enclosed in a capsule), in combination with charcoal, and vitamin B6, to treat hangover and reduce alcoholic syndrome.

International Patent Publication from Mizumoto et al discloses a composition of fermented citrus molasses and a plant worm extract that will promote alcohol metabolism and therefore reduce the sickness from drinking and hangover. Also, European Patent 1066835 to Kim proposes the use of extracts of leaves, stalks and fruits of pepino to lower blood alcohol concentration and reduce hangover.

Nonetheless, all the prior art methods and compositions for reducing health damages or drinking hangover have only limited effects. Therefore, there is a need for a composition and method that are effective in reducing health damages or drinking hangover.

The embodiments described below address this need.

### SUMMARY OF THE INVENTION

Described herein are methods drawn to accelerate the removal (metabolism) of ingested alcohol from the human body.

In one embodiment of the present invention, it is provided a composition and a method of use thereof for removing alcohol through accelerated metabolism of alcohol (ethanol) to acetate in the stomach and/or gastro-intestine prior its entrance to the circulation system, thereby preventing alcohol intoxication.

In another embodiment of the present invention, it is provided a composition and a method of use thereof for stimulating the activities of human alcoholases to increase the rate of alcohol oxidation in the body, which prevents the increase in blood alcohol concentration and alleviate drunkenness. Exemplary alcoholases include, but are not limited to, alcohol dehydrogenases, aldehyde dehydrogenases, alcohol oxidases, aldehyde oxidases, NADH oxidases, NADH dehydrogenases, and NADH oxidizing enzymes which utilize NADH as co-substrate.

In still another embodiment of the present invention, it is provided a composition and a method of use thereof for increasing or maintaining the concentration of coenzyme NAD and the NAD/NADH ratio (i.e., redox state) in the body, which enhances the alcohol metabolism rate and prevents the drinker from developing alcohol-drinking related diseases and alcoholic syndrome.

In a further embodiment of the present invention, it is provided a nutritional/pharmaceutical composition for accelerating the metabolism of alcohol and maintaining health redox states to prevent/reduce alcohol intoxication, drunkenness and hangover.

### BRIEF DESCRIPTION OF THE DRAWINGS

FIG. 1 lists various pathogenic effects of alcohol and its metabolites.

FIG. 2 is a scheme showing the procedures for preparation of animal glandular extract.

FIG. 3 shows the absorbance change measured at 500 nm for pig liver extract (Extract II) in the presence of INT only, INT+acetaldehyde, and INT+ethanol, respectively. Phosphate buffer: 0.05 M (pH=7.5); INT: 12.7 mM; ethanol: 1.0% (V/V); aldehyde: 17.6 mM. No NAD was added.

FIG. 4 shows the absorbance increase as a measure of acetate formation from ethanol oxidation by animal glandular extracts. Testing conditions: Tris Buffer (0.05 M), pH=8.5; alcohol concentration=3%; Extract 11=1.0 g in 100 ml solution.

FIG. 5 shows alcohol dehydrogenase activity as a function of initial NAD concentration. Testing conditions: 0.05 M Tris Buffer (pH=8.5); ethanol=3% (V/V); Alcohol dehydrogenase (from Sigma Aldrich Chemical Company): 5 units. ADH activity was determined by measuring absorbance change at 340 nm.

FIG. 6 is a schematic presentation of accelerating alcohol oxidation (metabolism) by coupling NAD regeneration reactions with diaphorases and INT.

FIG. 7 shows absorbance increase vs. time for alcohol dehydrogenase (prepared from yeast extract YSC-1, SigmaAldich) in the presence of 0.5 mM and 2.0 mM NAD, respectively. Phosphate buffer: 0.05 M (pH=7.5); ethanol: 1.0% (V/V). There was no absorbance change in the absence of NAD.

FIG. 8 shows absorbance change measured at 500 nm for alcohol dehydrogenase prepared from yeast extract (SigmaAldrich, YSC-1) in the presence of INT only, INT+ethanol, INT+ethanol+NAD, and INT+ethanol+NAD+diaphorase, respectively. Phosphate buffer: 0.05 M (pH=7.5); INT: 12.7 mM; ethanol: 1.0% (V/V); NAD=2.0 mM; Diaphorase: 2 units.

FIG. 9 shows stimulation of NADH oxidation in liver mitochondria by aspartate (A) and malate (B).

FIG. 10 shows stimulation of NADH oxidation by hormones with porcine liver extracts.

FIG. 11 shows stimulation of alcohol dehydrogenase by Mg2+.

FIG. 12 shows stimulation of alcohol dehydrogenase by various phosphates (IDP-inosine diphosphate, ADP-adenosine diphosphate; ATP-adenosine triphosphate).

FIG. 13 shows stimulation of alcohol dehydrogenase by diethylstilbestrol.

FIG. 14 shows thyroxine stimulation of aldehyde dehydrogenase activity.

FIG. 15 shows effect of administrating sodium pyruvate and alanine on the reduction rate of blood alcohol in dog. Dog body weight: 13.9 kilograms. 42 grams of alcohol was administrated at time 0. ●—Control (i.e., no further treatment in the first 5 hours); ▪—10 grams pyruvate was administrated orally at 5 hours; ▴ 10 grams of alanine was administrated at 1.5 hour and then 5 hours.

FIG. 16A shows the procedure for preparation of yeast extract; FIG. 16B shows the absorbance increase from ethanol oxidation by yeast extracts. Testing conditions: Tris Buffer (0.05 M), pH=8.5; alcohol concentration=3%.

### DETAILED DESCRIPTION

### Definitions

As used herein, the term alcohol refers to ethanol, ethanol-containing beverages or any substance that may be metabolized in vivo to generate ethanol.

The term nicotinamide adenine dinucleotide (NAD) is also known as diphosphate nucleotide (DPN). The term NADH refers to reduced NAD.

The term ADH refers to alcohol dehydrogenase, and ALDH refers to aldehyde dehydorgenase. The term Aox refers to alcohol oxidase. The term ALOx is short for aldehyde oxidase.

As used herein, the term NADH oxidizing enzymes refers to any enzymes that use NADH as the co-substrate and produce NAD as a co-product.

The term alcoholases refer to any of enzymes or combinations thereof that are involved in and/or related, directly or indirectly, to the metabolism of alcohol. Representative alcoholases include, but are not limited to, ADH, ALDH, AOx, ALOx, NADH oxidase, NADH dehydrogenases and combinations thereof.

As used throughout the description of the present application, alcohol refers to ethanol, and the term “alcohol” and the term “ethanol” are used interchangeably.

### Pharmaceutical or Nutraceutical Compositions That Accelerate Alcohol Metabolism

Provided herein are compositions and methods of making and using the same for accelerating metabolism of alcohol to reduce the level of blood alcohol. The compositions and methods of use thereof are provided to (a) accelerate the digestion and metabolism of alcohol in the gastric and/or gastrointestinal systems into acetate before the alcohol enter into the body's blood (i.e., circulation system), thereby preventing blood alcohol buildup and avoiding intoxication by alcohol; (b) reduce or eliminate the production and accumulation of toxic alcohol metabolites, such as acetaldehyde and free radicals, thus preventing hangover, reducing relapse, and protecting the liver and body organs from damaging by the toxins; and (c) maintain the human body at the healthy redox state.

In one aspect of the present invention, it is provided a composition for stimulating the activities of human alcoholases to increase the rate of alcohol oxidation in the body, which prevents the increase in blood alcohol concentration and alleviate drunkenness. Alcoholases can be any enzymes that are related to and/or involved in alcohol metabolism. Representative alcoholases include, but are not limited to, alcohol dehydrogenases, aldehyde dehydrogenases, alcohol oxidases, aldehyde oxidases, NADH oxidases, NADH dehydrogenases, other oxidizing enzymes that use NADH as co-substrate, and combinations thereof. In one embodiment, the composition may further include coenzymes NAD and its reduced form NADH, its precursors, nicotinamide, adenine, vitamin Bs, magnesium salts, pyrophosphate, nucleotide polyphosphates, hormonal substances, pyruvate, fructose, acetoacetate, adenosine diphosphate (ADP), adenosine triphosphate (ATP), adenosine monophosphate (AMP), amino acids that lead to NAD production, vitamin Ks, thyroxine and its analogues, and combinations thereof. In some embodiments, the hormonal substance can be, for example, diethylstilbestrol (DES), dehydroepi-androsterone (DHEA), estrone, androsterone, cortisone, testerone and combinations thereof. In some other embodiments, the amino acid can be any amino acids, exemplary of which are alanine, glutamine, argentine, aspartamine, aspartate, glutamate, tyrosine, leucine, lysine and combinations thereof. In some further embodiments, the thyroxine analogues can be, for example, 3,3,5-triiodo-thyronine, 3,5-diiodo-thyronine, 3,3′,5,5′-tetraiodo-thyropropionic acid, 3,3′,5-triiodo-thyropropionic acid, 3,3′,5′-triiodo-thyropropionic acid, 3,3′,5,5′-tetraiodo-thyroacetic acid, 3,3′,5-triiodo-thyroacetic acid, 3,3′,5′-triiodo-thyroacetic acid, 3,5-diiodo-thyroacetic acid, 3,5-diiodo-thyrosoine, and combinations thereof.

In a further aspect of the present invention, it is provided nutritional/pharmaceutical compositions for accelerating the metabolism of alcohol and maintaining health redox states to prevent/reduce alcohol intoxication, drunkenness and hangover. In one embodiment composition contains a substance that stimulates or activates an alcohol metabolizing enzyme in an amount effective to reduce alcohol intoxication and optionally a pharmaceutically or physiologically acceptable carrier. In some embodiments, the alcohol metabolizing enzyme can be, for example, alcohol dehydrogenase (ADH), aldehyde dehydrogenase (ALDH), alcohol oxidases, aldehyde oxidases, NADH oxidases, NADH dehydrogenases, and NADH oxidizing enzymes, and combinations thereof. In some other embodiments, the composition may further include a substance such as one of coenzymes NAD and its reduced form NADH, its precursors, vitamin Bs, magnesium salts, nucleotide polyphosphates, hormonal substances, and combinations thereof. In some further embodiments, the hormonal substance can be, for example, diethylstilbestrol (DES), dehydroepi-androsterone (DHEA), estrone, androsterone, cortisone, testerone and combinations thereof.

In a further aspect of the present invention, it is provided a composition for reducing blood level of alcohol capable of for increasing or maintaining the concentration of coenzyme NAD and the NAD/NADH ratio (i.e., redox state) in the body. In one embodiment, the composition contains NADH oxidation co-substrates and their precursors capable of promoting the regeneration of NAD that catalyzes the metabolism of alcohol and optionally a pharmaceutically or physiologically acceptable carrier, thereby reducing the drunkenness and prevents hangover. In another embodiment, the composition may further include a substance such as any of pyruvate, fructose, acetoacetate, ADP, ATP, AMP, amino acids that lead to NAD production, vitamin Ks, thyroxine and its analogues, and combinations thereof. In some embodiments, the amino acid can be alanine, glutamine, argentine, aspartamine, aspartate, glutamate, tyrosine, leucine, lysine, and combinations thereof. In some other embodiments, the thyroxine analogue can be 3,3,5-triiodo-thyronine, 3,5-diiodo-thyronine, 3,3′,5,5′-tetraiodo-thyropropionic acid, 3,3′,5-triiodo-thyropropionic acid, 3,3′,5′-triiodo-thyropropionic acid, 3,3′,5,5′-tetraiodo-thyroacetic acid, 3,3′,5-triiodo-thyroacetic acid, 3,3′,5′-triiodo-thyroacetic acid, 3,5-diiodo-thyroacetic acid, 3,5-diiodo-thyrosoine, and combinations thereof.

The enzymes, coenzymes and any other substances described herein are either commercially available or can be readily obtained from an organism such as plants, microbes such as bacteria or yeast or animal tissues in a purified form or as an extract. The extract can be, for example, an extract from yeast or an extract from an animal tissue such as animal liver, hearts, kidney, intestine, and stomach.

The compositions described herein can be used for example, for removal of alcohol through accelerated metabolism of alcohol to acetate prior its entrance to the human body circulation system, thereby preventing alcohol intoxication. In addition, the compositions defined herein can be used, for example, for increasing or maintaining the concentration of coenzyme NAD and the NAD/NADH ratio (i.e., redox state) in the body, which enhances the alcohol metabolism rate and prevents the drinker from developing alcoholic syndrome.

### Alcohol Metabolism

Alcohol metabolism requires one or more of alcohol oxidizing enzymes (alcoholases) together with coenzyme NAD. In the present invention, alcoholases refer to alcohol dehydrogenases (ADH), aldehyde dehydrogenases (ALDH), alcohol oxidases, aldehyde oxidases, NADH oxidases, NADH dehydrogenases, and NADH oxidizing enzymes (including sorbitol dehydrogenase, lactate dehydrogenase, diaphorase, NADH oxidases, and NADH dehydrogenases that use NADH as co-substrate for regeneration of NAD), and combinations thereof.

When alcohol beverages are taken, the alcohol enters the stomach and is soon transported to the small intestine. From here the alcohol enters the blood stream with water via the portal vein and goes to the liver and every part of the body through the general circulation. There are several routes of metabolism of alcohol in the body. Studies have shown that the alcohol metabolism enzymes are found in highest amount in the liver and only in a very small amount in the stomach mucosa. Because rapidly ingesting alcohol is quickly passed from the stomach into the duodenum, the major pathways of alcohol metabolism involve the liver. It is estimated that more than 90% of the alcohol that enters the body is metabolized in the liver.

The first step of alcohol metabolism involves ethanol oxidation by alcohol dehydrogenases (ADH) according to following reaction:

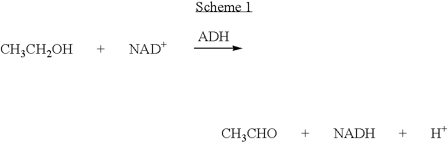

The enzyme alcohol dehydrogenase requires a coenzyme called nicotinamide adenine dinucleotide (NAD) in order to catalyze the oxidation of alcohol. This step of the metabolism produces acetaldehyde, which a highly toxic substance.

The second step is the oxidation of acetaldehyde to acetic acid, which is catalyzed by acetaldehyde dehydrogenase (ALDH): 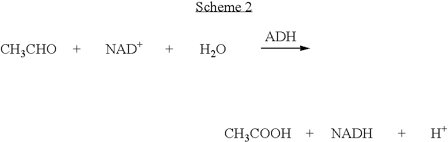
Effective removal of acetaldehyde is important not only to prevent cellular toxicity, but also to maintain efficient removal of ethanol, e.g., acetaldehyde is a product inhibitor of ADH. The balance between the various ADH and ALDH enzymes regulates the concentration of acetaldehyde, which is important as a key risk factor for the development of alcoholism. Chronic alcohol consumption decreases acetaldehyde oxidation, either due to decreased ALDH activity or to impaired mitochondrial function. As a result, circulating levels of acetaldehyde are usually elevated in alcoholics because of increased production, decreased removal, or both.

Alcohol metabolism in human follows the zero-order kinetics, that is, the reduction of alcohol concentration in blood proceeds at a constant rate, independent of the blood alcohol concentration. This shows that the two most important factors controlling the rate of alcohol metabolism in human are the total activity of alcohol dehydrogenase and the concentration of coenzyme NAD. It is reasonably expected that any means of stimulating the enzymes activity in the human body system will increase the rate of alcohol metabolism. Similarly, an increase in the concentration of NAD available for alcohol oxidation will increase the oxidation of alcohol.

### A. Use of Animal Glandular Extractions

In one aspect of the present invention, animal glandular extractions can be used to accelerate alcohol metabolism. The animal glandular extracts contain active alcoholases and coenzymes in either oxidized or reduced forms for example, NAD and/or NADH. Animal glandular extracts accelerate the metabolism of alcohol into harmless acetate in the gastrointestinal system before the alcohol enters into the body's circulation system.

In one embodiment of the present invention, it is provided a composition comprising an animal glandular extract, optionally with a pharmaceutically acceptable or physiologically acceptable carrier. The animal glandular extract can be prepared from any animal glandular part. The term animal glandular part refers to any animal organs, including liver, heart, kidney, stomach, intestine, pancreas, and combinations thereof.

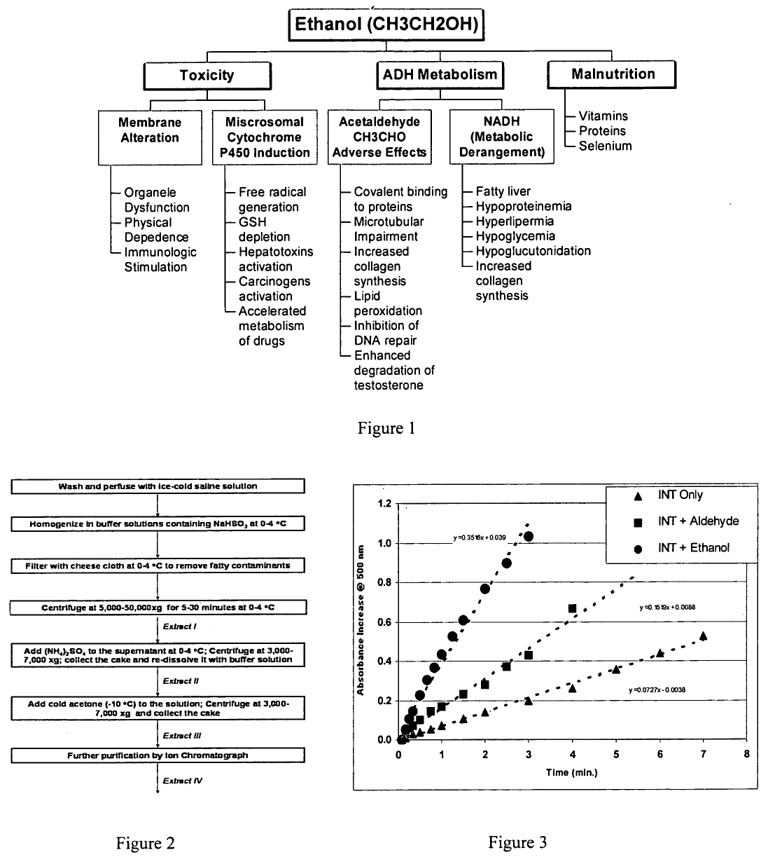

In one embodiment, a method has been developed to prepare animal glandular extracts that contain enzymes of specific activity for alcohol oxidation. An exemplary preparation process is shown schematically in FIG. 2 and described in Example 1, below.

### B. Coupling Regeneration of NAD

As shown in Schemes 1 and 2, above, a mechanism of alcohol metabolism by ADH and ALDH enzymes requires coenzyme NAD. The oxidation of each mole of ethanol consumes two moles of NAD. As NAD is being depleted, the redox ratio, i.e., NAD⁺/NADH ratio, decreases, so the oxidation of ethanol is restricted by the limited availability of NAD. NAD thus becomes a limiting factor of alcohol metabolism.

Accordingly, to maintain effective rates of ethanol oxidation by ADH, it is thus desirable to regenerate NAD⁺ from the NADH produced by the ADH reaction as shown in Schemes 1 and 2 such that the redox ratio, NAD⁺/NADH, maintains relatively unchanged.

In the human beings and other mammalian animals, the redox ratio, NAD⁺/NADH, is regulated by a number of enzymes, including lactate dehydrogenase, β-hydroxybutyrate dehydrogenase (β-HBDH), NADH oxidase (or dehydrogenase), and oxidative phosphorylation. For example, the redox ratio can be regulated by lactate dehydrogenase (LDH) in the cytosol through the following reaction (Scheme 3): 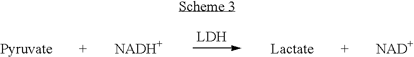
In mitochondria, the redox state is regulated by β-hydroxybutyrate dehydrogenase according to the reaction (Scheme 4):

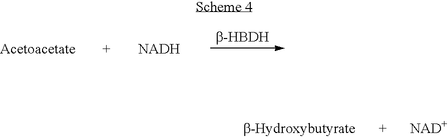

Under normal conditions, the blood concentration of cytosolic pyruvate is lowered quickly after ingestion of alcohol, therefore, regeneration NAD though oxidation of NADH in the cytosol is limited. The major system for converting NADH back to NAD is the mitochondrial electron transfer system, which converts NADH back to NAD via re-oxidization of NADH. However, because intact mitochondria are not permeable to NADH, it is necessary to transfer the reducing equivalents of NADH present in the cytosol into the mitochondria by substrate shuttle mechanisms. Therefore, the supply of NAD in the cytosol is governed by two factors: (a) the transfer of reducing equivalents into mitochondria (i.e., shuttle capacity of NADH); and (b) the capacity of the mitochondrial respiratory chain to oxidize these reducing equivalents (i.e., rate of oxidation of NADH). Shuttle capacity may become limiting under fasting metabolic states as the levels of shuttle components decrease, which lowers rates of ethanol oxidation.

A substrate for cytosolic enzymes can be used to maintain the ratio of NAD⁺/NADH in the cytosol. In one embodiment of the present invention, NAD in the cytosol can be regenerated by administering to a user composition comprising an effective quantity of substrate for cytosolic enzymes that use NADH as the cofactor and optionally a pharmaceutically or physiologically acceptable carrier to maintain the NAD⁺/NADH ratio by reducing the level of a substrate thereof. Such enzymes include, for example, lactate dehydrogenase (LDH), sorbitol dehydrogenase, β-hydroxybutyrate dehydrogenase, malate dehydrogenase, and diaphorase. As used herein, the term “an effective quantity” refers to a quantity of a substrate of an enzyme, which, upon administration to a user, is capable of regenerating about 1%, about 5%, about 10%, about 20%, about 25%, about 30%, about 40%, about 50%, about 60%, about 75%, about 80%, about 90%, about 95%, about 99%, about 100% of the NAD+that was used in the cytosol in metabolizing alcohol.

Alternately, the ratio of NAD⁺/NADH in the cytosol can be maintained by NADH shuttling by administering to a user a composition comprising an effective quantity of a substrate shuttle and optionally a pharmaceutically or physiologically acceptable carrier. The two major substrate shuttles are the α-glycerophosphate shuttle and the malate-aspartate shuttle. As used herein, the term “an effective quantity” refers to a quantity of a substrate of an enzyme, which, upon administration to a user, is capable of shuttling about 1%, about 5%, about 10%, about 20%, about 25%, about 30%, about 40%, about 50%, about 60%, about 75%, about 80%, about 90%, about 95%, about 99%, about 100% of the NADH that was generated in the cytosol in metabolizing alcohol.

In a further embodiment, the ratio of NAD⁺/NADH in the cytosol can be maintained by administering to a user an agent (for example, pure oxygen) that enhances re-oxidation of NADH by the respiratory chain.

### C. Enzyme Activators for Accelerating Alcohol Metabolism

In a further aspect of the present invention, a composition comprising a stimulator or activator compound that stimulates or activates an alcoholase and optionally a pharmaceutically or physiologically acceptable carrier can be administered to a user to accelerate alcohol metabolism. The compounds and their respective stimulation effects on the activities of alcoholases including alcohol dehydrogenases, aldehyde dehydrogenases and NAD regenerating enzymes are shown and described in Examples 9-15.

The compositions described herein can include any of the animal glandular extract, substrates of cytosolic enzymes, stimulators or activators and combinations thereof.

### Formulations

The composition can be formulated into any form suitable for a given mode of delivery to a user. For example, for oral delivery, the composition can be formulated into, for example, capsules, tablets, suspensions, liquid formulations. For parenteral administration or delivery, the composition can be a liquid or suspension in a pharmaceutically acceptable or physiologically acceptable carrier such as water.

The formulations can be administered to a user in need thereof via any of suitable mode of administration such as parenteral administration and oral administration. Preferably, the mode of administration is oral administration.

The embodiments of the present invention will be illustrated by the following set forth examples. All parameters and data are not to be construed to unduly limit the scope of the embodiments of the invention.

### EXAMPLES

### Example 1

### Preparation of Animal Glandular Extracts

Fresh animal glandular parts were cleaned and perfused with a liquid such as ice-cold saline and then homogenized with, for example, 4 volumes of an iced-cold buffer solution (0.1 M potassium phosphate, pH=7.8) and 1 mM sodium bisulfite using a kitchen homogenizer. All of the following procedures were performed at 0-4° C. and with the same buffer containing 0.1 mM sodium bisulfite. The homogenate was then centrifuged at about 20,000 g for 30 minutes. The supernatant (Extract I) was fractionated by adding ammonium sulfate (30-50% saturation) to obtain precipitate. The precipitate (Extract II) was separated by centrifuging at 5,000 g for 10 minutes. The precipitate was re-dissolved in a small volume of buffer. Cold acetone (−10° C.) was added to the solution to obtained precipitate (Extract III). The solid were then further purified by ion exchange chromatograph, gel-filtration, and/or affinity chromatograph, as needed (Extract IV).

A three-step procedure was used to prepare the alcohol metabolizing enzymes from 400 grams of pig liver. The purity and yield of the extract were determined based on the alcohol dehydrogenase activity. Typical results are given in Table 2.

TABLE 2
Purity and yield of alcohol dehydrogenases prepared from pig liver

| | | Total | Specific | | |
|---|---|---|---|---|---|
| | Total | ADH | ADH | | |
| Preparation | Protein | activity | Activity | Purification | Yield |
| Step | (mg) | (units) | (U/mg) | factor | (%) |
| Centrifuged | 5000 | 505 | 0.101 | 1.0 | 100 |
| Homogenate | | | | | |
| (Extract I) | | | | | |
| Ammonium | 2210 | 430 | 0.195 | 1.95 | 85 |
| Sulfate | | | | | |
| Precipitate | | | | | |
| (Extract II) | | | | | |
| Acetone | 1408 | 394 | 0.280 | 2.8 | 78 |
| Precipitate | | | | | |
| (Extract III) | | | | | |

### Example 2

### Enzyme Activity of Pig Liver Extracts

The animal glandular extracts prepared according to Example 1 were tested for a variety of enzyme activities: alcohol dehydrogenases, aldehyde dehydrogenases, lactate dehydrogenases, sorbitol dehydrogenases, and diaphorases, as described below.

a. Alcohol Dehydrogenase

The activity of alcohol dehydrogenases, which catalyzes the conversion of alcohol to aldehyde (Scheme 1), was determined by spectrophotometric assay method. Formation of acetaldehyde as shown in Scheme 1, supra, is favored by performing the reaction at pH=9 (e.g., in Tris or phosphate buffer) and coupling acetaldehyde formed with a trapping agent. NADH has a maximum absorbance at 340 nm. The unit of enzyme activity is defined as the absorbance increase (1 unit) per minute at 35° C. In each test, 3.0 ml of “ADH cocktail solution” containing glycine buffer reagent (Sigma-Aldrich No. 332-9, Sigma-Aldrich, St. Louis, Mo.), 1% ethanol (V/V), and 3.0 mM NAD. The change of absorbance at 340 nm was followed after the addition of 10 or 20 μl of the animal glandular extract. The reported activity was the average of at least 6 assays.

b. Acetaldehyde Dehydrogenase

The activity of aldehyde dehydrogenases, which catalyzes the conversion of aldehyde to acetate as shown in Scheme 2, was determined by spectrophotometric assay method. Formation of acetaldehyde is favored by performing the reaction at pH=9 (e.g., in Tris or phosphate buffer). NADH has a maximum absorbance at 340 nm. The unit of enzyme activity is defined as the absorbance increase (1 unit) per minute at 35° C. In each test, 3.0 ml of “ALDH cocktail solution” containing 0.05 M Tris-buffer (SigmaAldrich, St. Louis, Mo.), 25 mM aldehyde, and 3.0 mM NAD. The change of absorbance at 340 nm was followed after the addition of 10 or 20 μl of the animal glandular extract. The reported activity was the average of at least 6 assays.

C. Sorbitol Dehydrogenase

The activity of sorbitol dehydrogenases (SDH), which catalyzes the conversion of d-fructose to sorbitol (Scheme 5), was determined by spectrophotometric assay method. 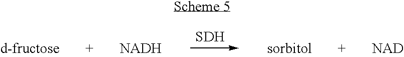
The reaction shown in Scheme 5 was performed at pH=7 (in Tris buffer). The unit of enzyme activity is defined as the absorbance decrease (1 unit) per minute at 35° C. In each test, 3.0 ml of “SDH cocktail solution” containing 0.05 M Tris-buffer (SigmaAldrich, St. Louis, Mo.), 50 mM d-fructose, and 0.3 mM NADH. The change of absorbance at 340 nm was followed after the addition of 10 or 20 μl of the animal glandular extract. The reported activity was the average of at least 6 assays.

d. Lactate Dehydrogenase

The activity of lactate dehydrogenases (LDH), which catalyzes the conversion of pyruvate to lactate (Scheme 6), was determined by spectrophotometric assay method. 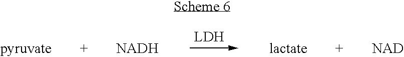
The reaction shown in Scheme 6 was performed at pH=7 (in Tris buffer). The unit of enzyme activity is defined as the absorbance decrease (1 unit) per minute at 35° C. In each test, 3.0 ml of “LDH cocktail solution” containing 0.05 M Tris-buffer (SigmaAldrich, St. Louis, Mo.), 50 mM sodium pyruvate, and 0.3 mM NADH. The change of absorbance at 340 nm was followed after the addition of 10 or 20 μl of the animal glandular extract. The reported activity was the average of at least 6 assays.

e. Malate Dehydrogenases

The activity of malate dehydrogenases (MDH), which catalyzes the conversion of oxalacetate to malate (Scheme 7), was determined by the following spectrophotometric assay method:

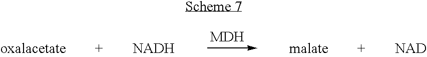

The reaction shown in Scheme 7 was performed at pH=7 (in Tris buffer). The unit of enzyme activity is defined as the absorbance decrease (1 unit) per minute at 35 ° C. In each test, 3.0 ml of “MDH cocktail solution” containing 0.05 M Tris-buffer (SigmaAldrich, St. Louis, Mo.), 50 mM sodium oxalacetate, and 0.30 mM NADH. The change of absorbance at 340 nm was followed after the addition of 10 or 20 μl of the animal glandular extract. The reported activity was the average of at least 6 assays.

f. Diaphorase

The activity of diaphorase (DPH) and other NADH oxidizing enzymes was determined by the following spectrophotometric assay method. 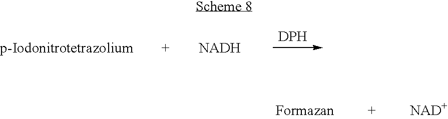
The reaction as shown in Scheme 8 was performed at pH=7 (in Tris buffer). Formazan has a maximum absorbance at 500 nm. The unit of enzyme activity is defined as the absorbance increase (1 unit) per minute at 35° C. In each test, 3.0 ml of “INT cocktail solution” containing 0.05 M Tris-buffer (SigmaAldrich, St. Louis, Mo.), 50 mM, and 0.3 mM NADH. The change of absorbance at 500 nm was followed after the addition of 10 or 20 μl of the animal glandular extract. The reported activity was the average of at least 6 assays.

An example is given here for pig liver extracts that exhibit high activities of several enzymes involved in the alcohol metabolism, as shown in the Table 3. It has been found that animal glandular extracts are rich in enzymes.

### Example 3

### Content of the Coenzyme NAD and its Reduced Form NADH in Animal Extracts

This example demonstrates that animal glandular extracts contain high levels of the coenzyme NAD and NADH that are required for alcohol metabolism. As described in testing method f, in the presence of diaphorases, INT is reduced to forzaman, while NADH is oxidized to NAD in a 1:1 mole stoichiometric ratio. Therefore, the increase in the absorbance at 500 nm is directly proportional to the concentration of forzaman.

FIG. 3 and Table 4 show the change of absorbance at 500 nm versus time. The results demonstrate that the animal glandular extract, prepared as described in example 1, contains high content of the coenzymes NADH. In the presence of INT, the oxidation of ethanol and acetaldehyde is enhanced substantially. Since NAD and NADH are high very difficult to purify, the costs of use of external high purity NAD has been proven to be prohibitively high. The present invention provides a cost-effective method for preparing the enzyme-coenzyme formulation.

TABLE 3
Enzyme activity of pig liver extracts

Specific Enzyme Activity (Unit/mg)

| Enzyme | Extract II | Extract III |
|---|---|---|
| Alcohol dehydrogenase | 0.195 | 0.280 |
| Aldehyde dehydrogenase | 0.364 | 0.502 |
| Sorbitol dehydrogenase | 0.137 | 0.195 |
| Lactate dehydrogenase | 0.100 | 0.130 |
| Diaphorase | 0.301 | 0.450 |

### Example 4

### Alcohol Metabolism by Animal Glandular Extract as Measured by Acetate Formation

The metabolism of alcohol produces acetate. The rate of formation of acetate is thus a measure of the alcohol metabolism rate. Acetate concentration is conventionally measured by an enzyme assay method that is based on acetate kinase and pyruvate kinase and change of NADH concentration. However, this method is not applicable when NADH and NAD coexist with acetate in the alcohol metabolism. In the present invention, a novel enzymatic method was developed to measure the acetate concentration in a solution that contains NADH, NAD and alcohol. The method is described as follows.

First, in the presence of coenzyme A (CoA) and adenosine triphosphate (ATP), acetate was converted by acetyl-CoA synthetase (ACS) to acetyl-CoA, producing adenosine monophosphate (AMP) and pyrophosphate (PPi) (Scheme 9). 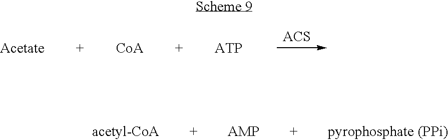
Second, pyrophosphate (PPi) was converted to phosphate (Pi) by inorganic pyrophosphatase (Scheme 10):

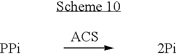

Third, maltose phosphorylase converts maltose to glucose-1-phosphate (G-1-P) and glucose (Scheme 11):

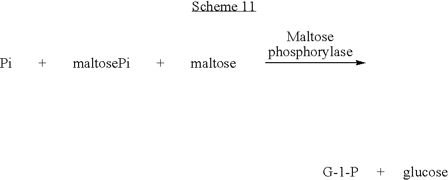

Fourth, the produced glucose is then converted to gluconic acid with glucose oxidase, with hydrogen peroxide as the co-product (Scheme 12):

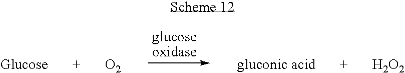

The quantity of hydrogen peroxide was then be measured with horse radish peroxidase (HRP) and dye per the reaction shown in Scheme 13.

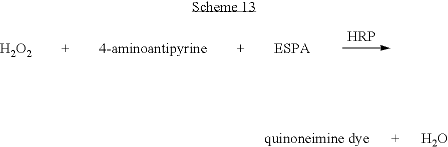

The quinoneimine dye has a maximum absorbance at 500-550 nm, depending on the specific dye used. The quantity of hydrogen peroxide is directly proportional to the acetate concentration, i.e., each mole of acetate will product a mole of hydrogen peroxide.

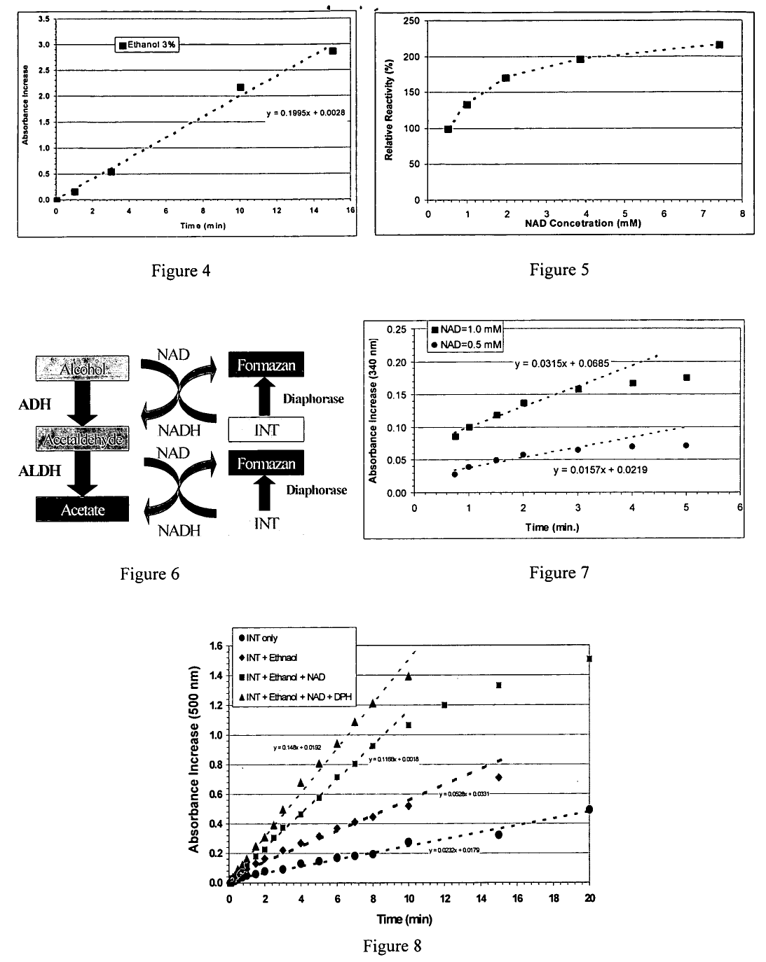

Results shown in FIG. 4 demonstrate that acetate formation increases linearly with time under the given conditions, indicating that alcohol metabolism to acetate follows a zero order kinetic law.

TABLE 4
Absorbance change without INT (measured at 340 nm) and with INT
(measured at 500 nm)*.

| | | Addition of |
|---|---|---|
| Substrate | No NAD Addition | NAD = 2.0 mM |
| Ethanol (340 nm) | 0.04 | 0.08 |
| Acetaldehyde (340 nm) | 0.04 | 0.128 |
| Ethanol + INT (500 nm) | 0.302 | 0.420 |
| Aldehyde + INT (500 nm) | 0.152 | 0.201 |

*Test conditions: Phosphate buffer: 0.05 M (pH = 7.5); ethanol: 1.0% (V/V) or aldehyde: 17.6 mM. In the absence of INT, alcohol oxidation testing solution contains aldehyde rapping agent.

### Example 5

### Dependence of Alcohol Dehydrogenase Activity vs. NAD Concentration

This example demonstrates two important aspects of alcohol oxidation: (a) the rate of alcohol oxidation depends on the NAD concentration; and (b) NAD can be used to accelerate alcohol metabolism. As shown in FIG. 5, alcohol oxidation by alcohol dehydrogenase increases with increasing NAD concentration.

### Example 6

### Regeneration of NAD Through Coupling Reactions

In this example, the INT system was selected to illustrate the effectiveness of the regeneration of NAD by coupling compounds, since the reaction product, i.e., formazan, gives a maximum absorbance at 500 nm. Then the coupling reactions (see FIG. 6) can be directly quantified by spectrophotometric measurements.

As shown in FIG. 7, addition of NAD increases the absorbance, demonstrating that alcohol is oxidized. The rate of absorbance increase is directly proportional to the initial NAD concentration. In the absence of NAD, there was no change in the absorbance, indicating that the extract contains no NAD.

FIG. 8 shows the absorbance change vs. time in the presence of INT. The absorbance increase in the presence of INT alone shows that the yeast extract contains significant level of NADH and diaphorase. In the presence of ethanol, the rate of absorbance increase is more than doubled, from 0.023 to 0.052 units/min. Addition of NAD and, particularly, together with diaphorase, yields a 100% to 200% increase in the rate of absorbance change. These results were similar to that observed with animal glandular extracts (see FIG. 3). Clearly, oxidation of ethanol is accelerated by the presence of INT and diaphorase.

### Example 7

### Stimulated NAD Regeneration by NADH Shuttling Enhancers

This example demonstrates the feasibility of accelerating the oxidation of NADH into NAD through using substances that stimulate the transfer of NADH into mitochondria, which in turn increases the metabolism of alcohol.

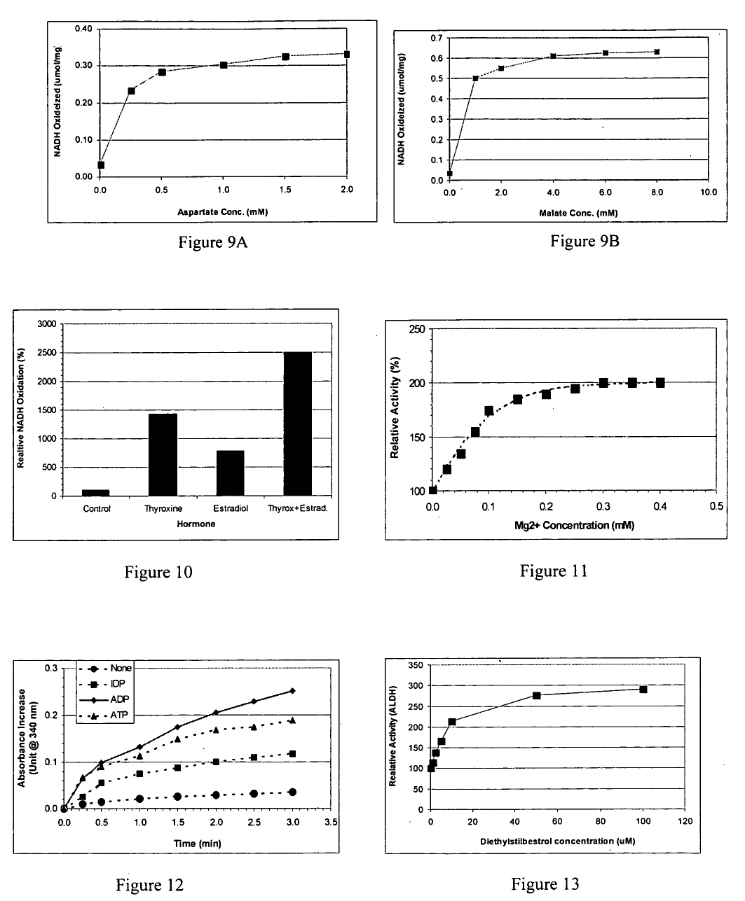

FIG. 9 shows the effect of added aspartate and malate on NADH oxidation catalyzed by the malate-aspartate shuttle in rabbit liver mitochondria. Addition of aspartate or malate substantially increases the oxidation of NADH. Malate is more effective than aspartate.

### Example 8

### NADH Oxidation Stimulated by Hormones

Several hormonal compounds were found to stimulate the oxidation of NADH, and thus the recycling of NAD. FIG. 10 compares the NADH oxidized in the presence of thyroxine and/or estradiol to that without hormone. When used alone, thyroxine and estradiol increased the oxidation of NADH by 5-15 times. Surprisingly, when used together, thyroxine and estradiol increased NADH oxidation by as much as times, indicating that the two hormonal compounds have strong synergistic effects on increasing NADH oxidation.

Many other compounds have been tested and found to stimulate the process of NADH oxidation, and thus the alcohol metabolism process. Table 6 gives a list of the substances and their chemical structure.

TABLE 5
Effect of thyroxine analogues on alcohol dehydrogenase activity

| | | Absorbance | |
|---|---|---|---|
| | | Change | Relative |
| | Dosage | (units/min | Increase |
| Thyroxine analogues | (μM) | @ 340 nm) | (%) |
| None | | 0.1 | 100 |
| L-thyroxine | 17 | 0.30 | 300 |
| 3,3,5-triiodo-l-thyronine | 17 | 0.446 | 446 |
| 3,3,5-triiodo-d-thyronine | 17 | 0.52 | 520 |
| 3,5-diiodo-l-thyronine | 17 | 0.148 | 148 |
| 3,5-diiodo-l-thyronine | 35 | 0.19 | 190 |
| 3,5-diiodo-l-thyronine | 70 | 0.275 | 275 |
| 3,5-dibromo-3′-iodo-thyronine | 17 | 0.48 | 480 |
| DL-thyronine | 17 | 0.535 | 535 |
| 3,3,5,5′-tetraiodothyropropionic acid | 17 | 0.75 | 750 |
| 3,3′5-triiodothyropropionic acid | 17 | 0.676 | 676 |
| 3,3′5′-triiodothyropropionic acid | 17 | 0.865 | 865 |
| 3,3′5,5′-tetraiodothyroacetic acid | 17 | 0.71 | 710 |
| 3,3′5-triiodothyroacetic acid | 17 | 0.715 | 715 |
| 3,5-diiodo-thyroacetic acid | 17 | 0.136 | 136 |
| 3,5-diiodo-thyroacetic acid | 70 | 0.238 | 238 |
| 3,5-diiodo-thyroacetic acid | 140 | 0.345 | 345 |
| 3,5-diiodo-l-thyrosine | 170 | 0.256 | 256 |
| 3,5-diiodo-l-thyrosine | 340 | 0.36 | 360 |

TABLE 6
Coupling compounds that stimulates NADH oxidation into NAD

| | | |
|---|---|---|
| Fructose | | |
| Synonyms Molecular Formula Molecular Weight CAS Number | Fructose, Fruit sugar D-Levulose C₆H₁₂O₆180.2 57-48-7 | 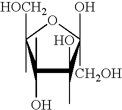 |
| Pyruvate salts | | |
| Synonyms Molecular Formula Molecular Weight CAS Number | Pyruvic acid sodium salt 2-Oxopropanoic acid sodium salt a-Ketopropionic acid sodium salt C₃H₃NaO₃110.0 113-24-6 | 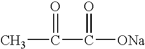 |
| Acetoacetate salts | | |
| Synonyms Molecular Formula Molecular Weight CAS Number | Acetoacetic acid lithium salt C₄H₅LiO₃108.0 3483-11-2 | 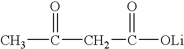 |
| Aspartate salts | | |
| Synonyms Molecular Formula Molecular Weight CAS Number | (S)-2-Amino- butanedioic acid sodium salt SodiumL-aspartate C₄H₆NNaO₄.H₂O 173.1 3792-50-5 | 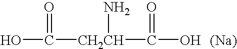 |
| Malate salt | | |
| Synonyms Molecular Formula Molecular Weight Synonyms | DL-Hydroxybutanedioic acid DL-hydroxysuccinic acid disodium C₄H₄Na₂O₅178.1 DL-Hydroxybutanedioic acid | 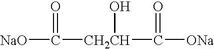 |
| Oxalacetic salts | | |
| Synonyms Molecular Formula Molecular Weight CAS Number | 2-Oxosuccinic acid Oxobutanedioic acid C₄H₄O₅132.1 328-42-7 | 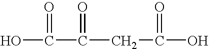 |
| Glutamate salts | | |
| Synonyms Molecular Formula Molecular Weight CAS Number | Potassium L-glutamate (S)-2-Aminopentanedioic acid L-□-Aminoglutaric acid potassium salt L-2-Aminopentanedioic acid potassium salt C₅H₈KNO₄.H₂O 203.2 19473-49-5 | 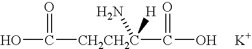 |
| dl-Alanine | | |
| Synonyms Molecular Formula Molecular Weight CAS Number | (±)-2-Aminopropionic acid C₃H₇NO₂89.09 302-72-7 | 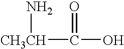 |

Iodonitrotetrazolium formazan

| | | |
|---|---|---|
| Synonyms Molecular Formula Molecular Weight CAS Number | INT-Formazan C₁₉H₁₄IN₅O₂471.3 7781-49-9 | 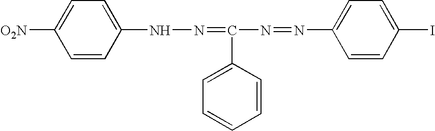 |

Menadione

| | | |
|---|---|---|
| Synonyms Molecular Formula Molecular Weight CAS Number | Vitamin K₃, 2-Methyl-1,4-naph- thoquinone C₁₁H₈O₂172.2 58-27-5 | 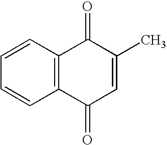 |

Water soluble Vitamin Ks

| | | |
|---|---|---|
| Synonyms Molecular Formula Molecular Weight CAS Number | 2-Methyl-3-phytyl-1,4-naph- oquinone 3-Phytylmenadione Phylloquinone Vitamin K₁₍₂₀₎C₃₁H₄₆O₂450.7 84-80-0 | 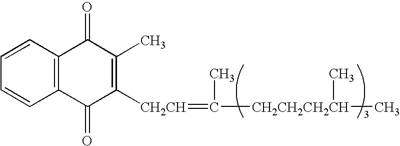 |

L-Thyroxine

| | | |
|---|---|---|
| Synonyms Molecular Formula Molecular Weight CAS Number | 3-[4-(4-hydroxy-3,5-di- ioodophenoxy)-3,5-di- iodophenyl]-L-ananine 3,3′,5,5′-Tetra- iodo-L-thyronine T₄C₁₅H₁₁I₄NO₄776.9 51-48-9 | 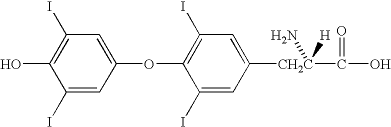 |

D-D-thyronine

| | | |
|---|---|---|
| Synonyms Molecular Formula Molecular Weight CAS Number | 3-[4-(4-Hydroxy-3,5-di- iodophenoxy)-3,5-di- iodophenyl]-D-alanine 3,3′,5,5′-Tetraiodo-D-thyronine C₁₅H₁₁I₄NO₄776.9 51-49-0 | 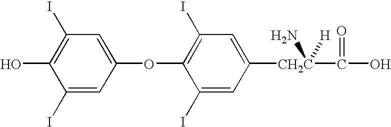 |

3,3′,5-Triiodo-L-thyronine (T₃)

| | | |
|---|---|---|
| Synonyms Molecular Formula Molecular Weight CAS Number | T₃O-(4-Hydroxy-3-iodo- phenyl)-3,5-diiodo-L-ty- rosine Liothyronine C₁₅H₁₂I₃NO₄651.0 6893-02-3 | 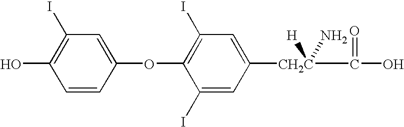 |

Reverse T₃

| | | |
|---|---|---|
| Synonyms Molecular Formula Molecular Weight CAS Number | Reverse T₃C₁₅H₁₂I₃NO₄651.0 5817-39-0 | 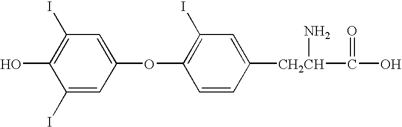 |

3,5-Diiodo-L-thyronine

| | | |
|---|---|---|
| Synonyms Molecular Formula Molecular Weight CAS Number | O-(4-Hydroxyphenyl)-3,5-di- iodo-L-tyrosine 3,5-Diiodo-4-(4-hy- droxyphenoxy)-L-phenyl- alanine C₁₅H₁₃I₂NO₄525.1 1041-01-6 | 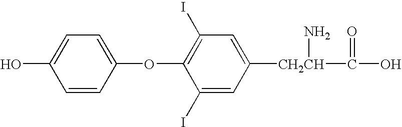 |

3,5-diiodothyroacetatic acid

| | | |
|---|---|---|
| Molecular Formula | C₁₄H₁₀I₂O₄ | 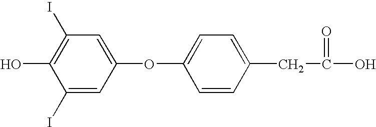 |

3,3′,5-Triiodothyroacetic acid

| | | |
|---|---|---|
| Synonyms Molecular Formula Molecular Weight CAS Number | 4-(4-Hydroxy-3-iodo- phenoxy)-3,5-di- iodophenylacetic acid C₁₄H₉I₃O₄621.9 51-24-1 | 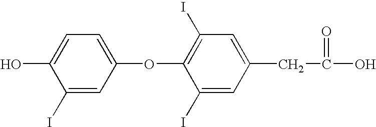 |

3,3′,5,5′-Tetraiodothyroacetic acid

| | | |
|---|---|---|
| Synonyms Molecular Formula Molecular Weight CAS Number | 4-(4-Hydroxy-3,5-di- iodophenoxy)-3,5-di- iodobenzeneacetic acid Tetrac C₁₄H₈I₄O₄747.8 67-30-1 | 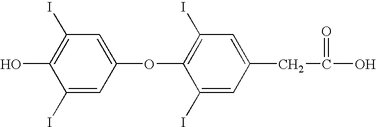 |

3,5-DiIodo-DL-Tyrosine

| | | |
|---|---|---|
| Molecular Formula CAS Number | C₉H₉I₂NO₃66-02-4 | 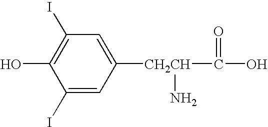 |

3-Iodo-L-tyrosine

| | | |
|---|---|---|
| Synonyms Molecular Formula Molecular Weight CAS Number | 3-Monoiodo-L-tyrosine C₉H₁₀INO₃307.1 70-78-0 | 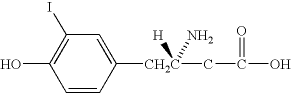 |

### Example 9

### Stimulating Effects of Magnesium Salts on Alcohol Enzymes

Magnesium salts was found to have strong activating effects on alcohol and aldehyde dehydrogenases. As shown in FIG. 11, the alcohol dehydrogenase activity can be increased by 100% at relatively high magnesium ion concentration.

### Example 10

### Enzyme Stimulation by Nucleotide Polyphosphates and Coenzymes

Polyphosphate compounds, such as inorganic pyrophosphate (PPi), adenosine monophosphate (AMP), adenosine diphopshate (ADP) and adenosine triphosphate (ATP). Typical experimental results are given in FIG. 12 for alcohol dehydrogenase stimulation by polyphosphate nucleotides. ADP showed the highest stimulating activity among all the polyphosphates tested.

Vitamin Bs and the precursors for coenzyme NAD are also found to have stimulating effects on alcohol oxidizing enzyme activity.

### Example 11

### Stimulation of Alcohol Oxidation Enzymes by Hormonal Compounds

Numerous hormonal compounds have been found to have stimulating effects on alcohol enzyme's activity. FIG. 13 shows the relative enzyme activity of alcohol dehydrogenase in the presence of various concentration of diethylstilbestrol. The enzyme's activity was increased by more than 100% by as low as 10 μM diethylstilbestrol. Similar stimulating effects were found with other hormonal compounds, as shown in Table 7.

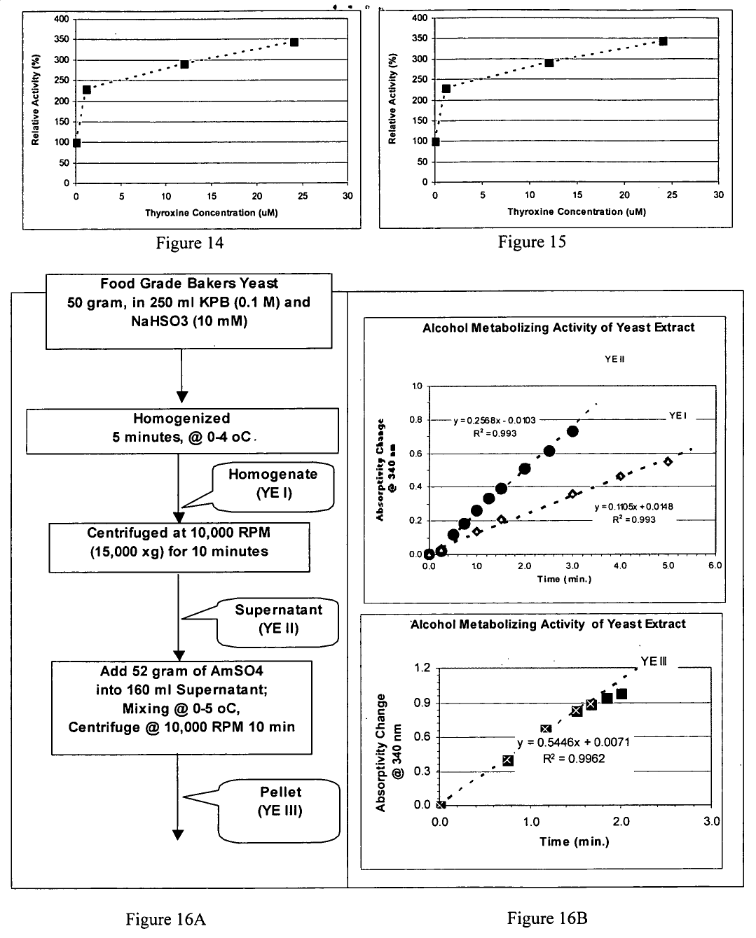

Thyroxine has extremely high stimulating effect on the enzyme activity of alcohol dehydrogenases and aldehyde dehydrogenases. As shown in FIG. 14, as little as 2 μM thyroxine increased the enzyme activity by more 100%, and the stimulation increased with increasing thyroxine concentration. Thyroxine analogues have all been found to have high stimulating effects on alcohol oxidizing enzymes, as shown in Table 5. Some are as twice effective as thyroxine at the same concentration. Surprisingly, the activation of alcohol and aldehyde dehydrogenases by thyroxine is significantly only in low doses. At high dosages, thyroxine becomes an inhibitor of the enzymes.

TABLE 7
Effect of hormonal compounds (hydroxyl steroids) on enzyme activity
of alcohol dehydrogenase.

| Compounds Name | Relative activity (%) |
|---|---|
| Control | 100.0 |
| Diethylstilbestrol | 259 |
| Dehydroepi-androsterone | 200 |
| Estrone | 156 |
| Androsterone | 119 |
| Cortisone | 116 |
| Progesterone | 120 |
| Deoxycorticosterone | 115 |
| Testerone | 137 |
| 4-Androsterone-3,17-dione | 128 |
| Corticosterone | 112 |

TABLE 8
Substances that stimulate alcohol oxidation enzyme activity and alcohol metabolism

Pyrophosphate salts

| Synonyms | Disodium pyrophosphate | Na₂H₂P₂O₇ |
|---|---|---|
| | Sodium diphosphate dibasic | |
| | Disodium dihydrogen | |
| | pyrophosphate | |
| Molecular Formula | H₂Na₂O₇P₂ | |
| Molecular Weight | 221.9 | |
| CAS Number | 7758-16-9 | |

Triphosphate salts

| | | |
|---|---|---|
| Synonyms | Pentasodium tripolyphosphate | Na₅P₃O₁₀ |
| Molecular Formula | Na₅O₁₀P₃ | |
| Molecular Weight | 367.9 | |
| CAS Number | 7758-29-4 | |

Adenosine

| | | |
|---|---|---|
| Synonyms Molecular Formula Molecular Weight CAS Number | Adenine-9-□-D-ri- bofuranoside Adenine riboside 9-□-D-Ribofuranosyladenine C₁₀H₁₃N₅O₄267.2 58-61-7 | 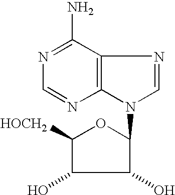 |

Adenosine monophosphate (AMP)

| | | |
|---|---|---|
| Synonyms Molecular Formula Molecular Weight | 2′-Adenylic acid 2′-AMP C₁₀H₁₄N₅O₇P 347.2 | 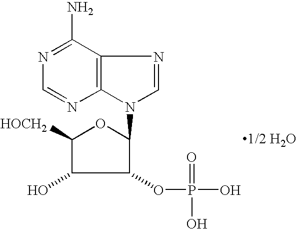 |

Adenosine 2′-monophosphate

| | | |
|---|---|---|
| Synonyms Molecular Formula | 3′-Adenylic acid 3′-AMP Adenosine 3′-mono- phosphoric acid C₁₀H₁₄N₅O₇P | 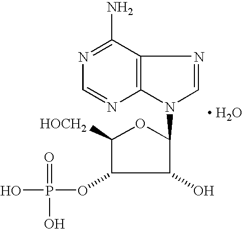 |

Adenosine 3′-monophosphate

| | | |
|---|---|---|
| Synonyms Molecular Formula Molecular Weight | 5′-AMP-Na₂C₁₀H₁₂N₅Na₂O₇P 391.2 | 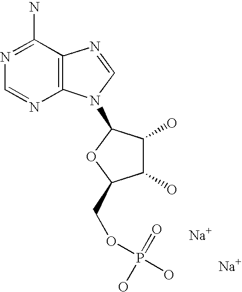 |

Adenosine 5′-monophosphate disodium salt
Adenosine diphosphate (ADP)

| | | |
|---|---|---|
| Synonyms Molecular Formula Molecular Weight CAS Number | ADP C₁₀H₁₄KN₅O₁₀P₂.2H₂O 501.3 72696-48-1 | 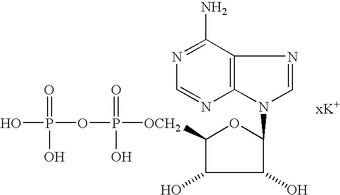 |

Adenosine Triphosphate (ATP)

| | | |
|---|---|---|
| Synonyms Molecular Formula Molecular Weight | 5′-ATP-K₂C₁₀H₁₄K₂N₅O₁₃P₃.2H₂O 619.4 | 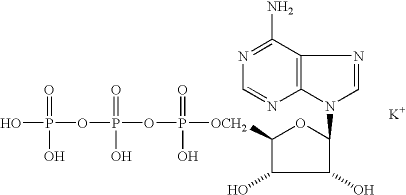 |

Diethylbestrol

| | | |
|---|---|---|
| Synonyms Molecular Formula Molecular Weight CAS Number | (E)-3,4-Bis(4-hy- droxyphenyl)-3-hex- ene Stilbestrol, DES C₁₈H₂₀O₂268.4 56-53-1 | 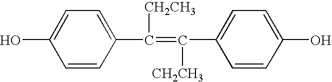 |

4-androstene

| | | |
|---|---|---|
| Synonyms Molecular Formula Molecular Weight CAS Number | 4-Androsten-17□-ol-3-one 17□-Hydroxy-4-androsten-3-one 17□-Hydroxy-3-oxo-4-an- drostene trans-Testosterone C₁₉H₂₈O₂288.4 58-22-0 | 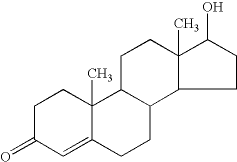 |

testosterone

| | | |
|---|---|---|
| Synonyms Molecular Formula Molecular Weight CAS Number | Methyl testosteronum 17□-Methyl-4-androsten-17□-ol-3-one 17□-Hydroxy-17□-methyl-4-an- drosten-3-one Mesterone; Methyltestosterone C₂₀H₃₀O₂302.5 58-18-4 | 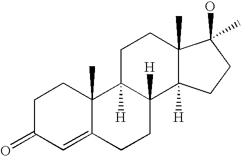 |

Progesterone

| | | |
|---|---|---|
| Synonyms Molecular Formula Molecular Weight CAS Number | 4-Pregnene-3,20-dione C₂₁H₃₀O₂314.5 57-83-0 | 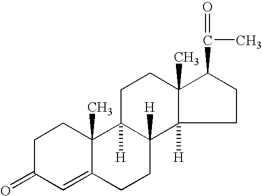 |

Epinephrine

| | | |
|---|---|---|
| Synonyms Molecular Formula Molecular Weight CAS Number | (−)-Adrenalin L-Adrenaline L-Epinephrine C₉H₁₃NO₃183.2 51-43-4 | 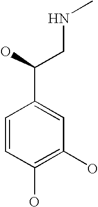 |

Deoxyepinephrine hydrochloride

| | | |
|---|---|---|
| Synonyms Molecular Formula Molecular Weight CAS Number | Epinine hydrochloride N-Methyldopamine hydrochloride 4-[2-(Methylamino)ethyl]pyro- catechol hydrochloride C₉H₁₃NO₂.HCl 203.7 62-32-8 | 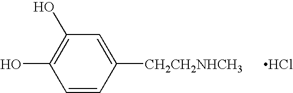 |

Norepinephrine

| | | |
|---|---|---|
| Synonyms Molecular Formula Molecular Weight CAS Number | Levarterenol (R)-4-(2-Amino-1-hy- droxyethyl)-1,2-benzenediol L-Noradrenaline L-Arterenol C₈H₁₁NO₃169.2 51-41-2 | 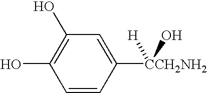 |

Estradiol

| | | |
|---|---|---|
| Synonyms Molecular Formula Molecular Weight CAS Number | 3,17□-Dihydroxy-1,3,5(10)-estra- triene 1,3,5-Estratriene-3,17□-diol Dihydrofolliculin 17□-Estradiol C₁₈H₂₄O₂272.4 50-28-2 | 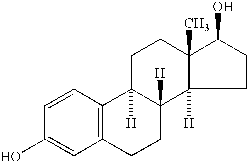 |

Nicotinamide Adenine Dinucleotide (NAD)

| | | |
|---|---|---|
| Synonyms Molecular Formula Molecular Weight CAS Number | Nadide Cozymase □-DPN DPN Coenzyme 1 □-NAD NAD Diphosphopyridine nucleotide C₂₁H₂₆N₇NaO₁₄P₂685.4 20111-18-6 | 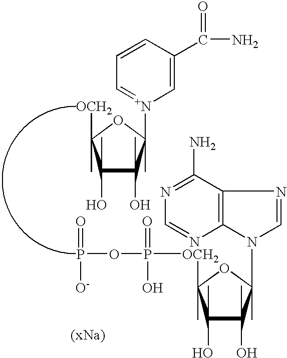 |

Nicotinamide

| | | |
|---|---|---|
| Synonyms Molecular Formula Molecular Weight CAS Number | Nicotinic acid amide Pyridine-3-carboxylic acid amide Niacinamide; Vitamin P, Vitamin B3 C₆H₆N₂O 122.1 98-92-0 | 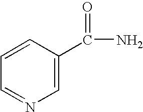 |

Vitamins Bs (Adenine, Vitamin B4)

| | | |
|---|---|---|
| Synonyms Molecular Formula Molecular Weight CAS Number | 6-Aminopurine Vitamin B₄C₅H₅N₅135.1 73-24-5 | 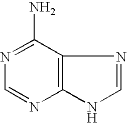 |

Vitamin H

| | | |
|---|---|---|
| Synonyms Molecular Formula Molecular Weight CAS Number | Vitamin H₁Vitamin BₓPABA 4-Aminobenzoic acid C₇H₇NO₂137.1 150-13-0 | 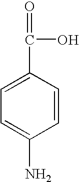 |

Nicotinic acid

| | | |
|---|---|---|
| Synonyms Molecular Formula Molecular Weight CAS Number | Acidum nicotinicum Pellagra preventive factor 3-Picolinic acid Pyridine-3-carboxylic acid Niacin; Vitamin B3 C₆H₅NO₂123.1 59-67-6 | 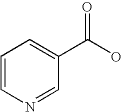 |

Pyridoxol

| | | |
|---|---|---|
| Synonyms Molecular Formula Molecular Weight CAS Number | Pyridoxol Vitamin B₆C₈H₁₁NO₃169.2 65-23-6 | 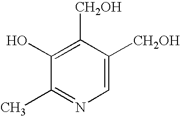 |

Vitamin B₁ hydrochloride

| | | |
|---|---|---|
| Synonyms Molecular Formula Molecular Weight CAS Number | Vitamin B₁ hydrochloride Aneurine hydrochloride C₁₂H₁₇ClN₄OS.HCl 337.3 67-03-8 | 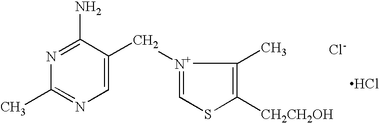 |

### Example 12

### In Vivo Tests of Alcohol Metabolism by Dog

In this example, the effectiveness of using compositions described above for accelerating alcohol metabolism was validated by in vivo tests with dogs. Six dogs (as described in Table 9) were used in the in vivo tests. The dogs were fasted 12 to 16 hours and then given 20% alcohol orally in a dose of 3 g/kg body weight. Approximately 1.5 hours were allowed for complete absorption and distribution of the alcohol. Blood samples were taken from the leg veins. The blood alcohol reading (BAR) was determined by measuring alcohol concentration in blood using the Sigma Aldrich alcohol assay method (Assay No. 333).

TABLE 9
Effect of oral administration of sodium pyruvate on blood alcohol
reading (BAR) in dog

| | | | | BAR | Sodium | | BAR | Sodium | | BAR |
|---|---|---|---|---|---|---|---|---|---|---|
| | | Body | Ethanol | Reduction | Pyruvate | Dosing | Reduction | Pyruvate | Dosing | Reduction |
| | | weight | Dose | Rate | Dose | Time | Rate | Dose | Time | Rate |
| Dog | Sex | (kg) | (g) | (ppm/hr) | (g) | (min) | (ppm/hr) | (g) | (min.) | (ppm/hr) |
| A | F | 9.0 | 27 | 81 | 5 | 266 | 252 | 5 | 320 | 195 |
| B | M | 10.8 | 32.5 | 67 | 10 | 404 | 272 | 10 | 464 | 256 |
| C | M | 13.9 | 42 | 60 | | | | | | |
| C | M | 13.9 | 42 | 66 | 10 | 250 | 218 | 10 | 310 | 230 |
| C | M | 13.9 | 42 | 82 | 5 | 400 | 212 | 5 | 460 | 215 |
| D | M | 22.0 | 60 | 77 | 10 | 405 | 113 | 5 | 465 | 109 |
| E | F | 13.5 | 40 | 84 | 5 | 410 | 160 | 5 | 470 | 157 |
| F | M | 12.3 | 37 | 95 | 5 | 376 | 253 | 5 | 436 | 223 |

| | | | |
|---|---|---|---|
| Average | 76.5 | 211.4 | 197.9 |

The effect of administrating pyruvate and alanine on the reduction of blood alcohol readings is summarized in Table 9. FIG. 15 illustrates typical blood alcohol vs. time curves. The well known linear decrease in blood alcohol was observed in the control curve. Following the oral administration of pyruvate or alanine, the reduction rate of BAR was increased by about 300%-400%, demonstrating that pyruvate and alanine strongly stimulate the metabolism of alcohol in the dogs.

### Example 13

### Composition Having a Yeast Extract for Facilitating Alcohol Metabolization

Food grade baker's yeast (50 g) was suspended in a 250 mL solution of potassium phosphate buffer (KPB) (0.1 M) and NaHSO₃ (0.01 mM) and then homogenized at 0-4° C. for 5 minutes. The homogenate (designated as YE I) was centrifuged for 10 minutes at 10,000 RPM (15,000×g). The resultant supernatant was designated as YE II.

To a 160 mL supernatant (produced as described above) was added 52 g of ammonium sulfate (AmSO4). The mixture was subjected to mixing at 0-5° C. and was then centrifuged at 10,000 RPM for 10 minutes. The resulted supernatant was allowed to concentrate to form into pellets, which was designated as YE III (FIG. 16A).

A sample for each of the YE I, YE II, and YE III was tested for facilitating alcohol metabolization. The test result was shown in FIG. 16B, showing that all of YE I, YE II, and YE III are effective in speeding up alcohol metabolization.

While particular embodiments of the present invention have been shown and described, it will be obvious to those skilled in the art that changes and modifications can be made without departing from this invention in its broader aspects. Therefore, the appended claims are to encompass within their scope all such changes and modifications as fall within the true spirit and scope of this invention.

1. A method for reducing blood level of alcohol, comprising: administering to a user a composition comprising an extract in an amount effective to reduce alcohol intoxication,
wherein the extract is selected from the group consisting of an animal glandular extract, a yeast extract, and a combination thereof.

2. A method for reducing blood level of alcohol, comprising: administering to a user a composition comprising a substance that stimulate or activate an alcohol metabolizing enzyme in an amount effective to reduce alcohol intoxication, wherein the alcohol metabolizing enzyme is selected from the group consisting of alcohol dehydrogenase (ADH), aldehyde dehydrogenase (ALDH), NADH oxidizing enzymes, and combinations thereof.

3. A method for reducing blood level of alcohol, comprising: administering to a user a composition comprising a substance selected from the group consisting of NAD, NADH oxidation co-substrates, precursors thereof, and combinations thereof to promote the regeneration of NAD that catalyzes the metabolism of alcohol, thereby reducing the drunkenness and prevents hangover.

4. The method of claim 1 wherein the animal glandular extract is an extract from an animal organ selected from the group consisting of animal liver, hearts, kidney, intestine, stomach and combinations thereof.

5. The method of claim 1 wherein the yeast extract is an extract from Saccharomyces cerevisiae.

6. The method of claim 2 wherein the composition further includes a substance selected from the group consisting of coenzymes NAD and its reduced form NADH, its precursors, nicotinamide, adenine, vitamin Bs, magnesium salts, pyrophosphate, nucleotide polyphosphates, hormonal substances, and combinations thereof.

7. The method of claim 6 wherein the hormonal substance is selected from the group consisting of diethylstilbestrol (DES), dehydroepi-androsterone (DHEA), estrone, androsterone, cortisone, testerone and combinations thereof.

8. The method of claim 3 wherein the composition further includes a substance selected from the group consisting of pyruvate, fructose, acetoacetate, adenosine diphosphate (ADP), adenosine triphosphate (ATP), adenosine monophosphate (AMP), amino acids that lead to NAD production, vitamin Ks, thyroxine and its analogues, and combinations thereof.

9. The method of claim 8 wherein an amino acid is selected from the group consisting of alanine, glutamine, argentine, aspartamine, aspartate, glutamate, tyrosine, leucine, lysine and combinations thereof.

10. The method of claim 8 where in an thyroxine analogue is selected from the group consisting of 3,3,5-triiodo-thyronine, 3,5-diiodo-thyronine, 3,3′,5,5′-tetraiodo-thyropropionic acid, 3,3′,5-triiodo-thyropropionic acid, 3,3′,5′-triiodo-thyropropionic acid, 3,3′,5,5′-tetraiodo-thyroacetic acid, 3,3′,5-triiodo-thyroacetic acid, 3,3′,5′-triiodo-thyroacetic acid, 3,5-diiodo-thyroacetic acid, 3,5-diiodo-thyrosoine, and combinations thereof.

11. A composition for reducing blood level of alcohol, comprising an extract in an amount effective to reduce alcohol intoxication and optionally a pharmaceutically or physiologically acceptable carrier,
wherein the extract is selected from the group consisting of an animal glandular extract, a yeast extract, and a combination thereof.

12. A composition for reducing blood level of alcohol, comprising a substance that stimulate or activate an alcohol metabolizing enzyme and optionally a pharmaceutically or physiologically acceptable carrier,
wherein the alcohol metabolizing enzyme is selected from the group consisting of alcohol dehydrogenase (ADH), aldehyde dehydrogenase (ALDH), alcohol oxidases, aldehyde oxidases, NADH oxidases, NADH dehydrogenases, and NADH oxidizing enzymes, and combinations thereof, in an amount effective to reduce alcohol intoxication.

13. A composition for reducing blood level of alcohol, comprising NADH oxidation co-substrates and their precursors capable of promoting the regeneration of NAD that catalyzes the metabolism of alcohol and optionally a pharmaceutically or physiologically acceptable carrier, thereby reducing the drunkenness and prevents hangover.

14. The composition of claim 11 wherein the animal extract is an extract from an animal tissue selected from the group consisting of animal liver, hearts, kidney, intestine, stomach, and combinations thereof.

15. The composition of claim 12 wherein the composition further includes a substance selected from the group consisting of coenzymes NAD and its reduced form NADH, its precursors, vitamin Bs, magnesium salts, nucleotide polyphosphates, hormonal substances, and combinations thereof.

16. The composition of claim 15 wherein the hormonal substance is selected from the group consisting of diethylstilbestrol (DES), dehydroepi-androsterone (DHEA), estrone, androsterone, cortisone, testerone and combinations thereof.

17. The composition of claim 13 wherein the composition further includes a substance selected from the group consisting of pyruvate, fructose, acetoacetate, ADP, ATP, AMP, amino acids that lead to NAD production, vitamin Ks, thyroxine and its analogues, and combinations thereof.

18. The composition of claim 17 wherein the amino acid is selected from the group consisting of alanine, glutamine, argentine, aspartamine, aspartate, glutamate, tyrosine, leucine, lysine, and combinations thereof.

19. The composition of claim 17 wherein the thyroxine analogue is selected from the group consisting of 3,3,5-triiodo-thyronine, 3,5-diiodo-thyronine, 3,3′,5,5′-tetraiodo-thyropropionic acid, 3,3′,5-triiodo-thyropropionic acid, 3,3′,5′-triiodo-thyropropionic acid, 3,3′,5,5′-tetraiodo-thyroacetic acid, 3,3′,5-triiodo-thyroacetic acid, 3,3′,5′-triiodo-thyroacetic acid, 3,5-diiodo-thyroacetic acid, 3,5-diiodo-thyrosoine, and combinations thereof.

21. The composition of claim 11 in an oral formulation.

22. The composition of claim 12 in an oral formulation.

23. The composition of claim 13 in an oral formulation.

24. The composition of claim 11 wherein the yeast extract is an extract from Saccharomyces cerevisiae.

25. A method of preparing a composition comprising a substance for reducing alcohol intoxication, comprising:
providing a substance in an amount effective to reduce alcohol intoxication; and
forming the composition,
wherein the substance is selected from the group consisting of an animal glandular extract, a yeast extract, a substance that stimulates or activates an alcohol metabolizing enzyme, a NADH oxidizing enzyme co-substrate or a precursor thereof, and combinations thereof.
